(19) United States
(12) Patent Application Publication
(10) Pub. No.: US 2023/0090569 A1
(43) Pub. Date: Mar. 23, 2023

(54) KITS AND METHODS FOR EXTRACTING NUCLEIC ACIDS FROM COMPLEX SAMPLES KITS AND METHODS FOR EXTRACTING NUCLEIC ACIDS FROM COMPLEX SAMPLES

(71) Applicant: ENALEES, Évry (FR)

(72) Inventors: Laurine VALOT, Évry (FR); Dove CORMIER, Évry (FR); Thomas THIBAULT, Évry (FR); Mégane SIMONNET, Évry (FR); Viviane LUONG, Évry (FR); Delphine SCHIEB, Évry (FR); Camille LAMARCHE, Évry (FR); Laurent THIERY, Évry (FR)

(73) Assignee: ENALEES, Évry (FR)

(21) Appl. No.: 17/799,084

(22) PCT Filed: Feb. 12, 2021
(86) PCT No.: PCT/EP2021/053534

§ 371 Date: Aug. 11, 2022

(30) Foreign Application Priority Data
Feb. 14, 2020 (FR) FR2001479

(51) Int. Cl. C12Q 1/6806

(52) U.S. Cl. CPC C12Q 1/6806

(57) ABSTRACT

A kit and method for extracting nucleic acids from complex samples. The kit includes: (i) a lysis buffer having a concentration of SDS ranging from about 1% to about 25%; (ii) a buffer having a concentration of a potassium salt of ranging from about 0.1 M to about 5.0 M; (iii) a buffer having a concentration of a zinc and/or copper salt of ranging from about 0.5 M to about 5.0 M; (iv) a filter having a pore diameter ranging from about 1 μm to about 10 μm; and optionally, a member selected from one or more syringe(s), one or more reaction tube(s), an instruction guide, and any combination thereof.

### FIELD OF INVENTION

The present invention relates to the field of nucleic acid extraction. More particularly, the invention relates to a kit and a method allowing the extraction of nucleic acids from complex samples, in a rapid and inexpensive manner, without the need to carry out tedious purification steps and without requiring a sophisticated equipment and neither suitable dedicated environment context, such as a laboratory.

### BACKGROUND OF INVENTION

The characterization of nucleic acids from a sample collected from a particular environment provides a valuable information about the nature of that environment. For example, advances in high throughput sequencing have recently allowed providing more efficient diagnosis of genetic diseases or infectious diseases. Indeed, precise knowledge of genetic information allows providing a targeted therapeutic approach. These advances also make it possible to identify species inhabiting a given biotope with great efficiency.

As a general rule, nucleic acids are contained in cells, viruses, parasites, and are therefore protected from external attacks by more or less sophisticated and robust membrane systems or wall systems. Thus, current techniques for analyzing nucleic acids necessarily require a first extraction step, i.e., the separation of nucleic acids from other cellular components. In practice, this involves lysing the membranes or cell walls to release nucleic acids, which can then be purified from other cellular components and/or other components of the original environment.

Several methods of nucleic acid extraction have been developed in the past, using both physical lysis processes, such as pressure or ultrasound, as well as chemical lysis processes. A frequently used chemical lysis method is based on the use of sodium dodecyl sulfate (or SDS), a strong ionic detergent and surfactant, which is usually combined with proteases for even more efficient lysis. This chemical method based on the use of SDS allows membranes or walls to burst and to release nucleic acids. For example, extraction methods based on lysis by SDS are used in particular in various fields, such as the study of seabed biomass (Natarajan et al.; Front. Microbiol., 2016, 7: 986), or the extraction of bacterial plasmidic DNA (principle of “mini-preparation”; see for example Green and Sambrook (Molecular Cloning: A Laboratory Manual; 4^{th} edition; Cold Spring Harbor Laboratory Press; 2012). However, the presence of SDS often raises a problem during the subsequent nucleic acid analysis steps. In particular, SDS is known to be an inhibitor of amplification by PCR (“Polymerase Chain Reaction”). Thus, specific extraction methods have been developed to be compatible with subsequent PCR steps (see, e.g., Goldenberger et al.; Genome Res., 1995, 4: 368-370).

Illustratively, patent application WO 01/34844 disclosed a method for isolating DNA from proteinaceous medium, including the formation of DNA-metallic oxide complex. Patent application WO 2015/120447 provides a method for nucleic acid purification.

In practice, SDS-based extraction methods almost always include nucleic acid purification steps. Purification can be done on affinity or exclusion columns, and/or alternatively, using a phenol/chloroform mixture or other chemicals. Alternatively or simultaneously, chemical compounds such as acetamide, betaine, bovine serum albumin, dextran, DMSO, Tween, etc., may be incorporated into the reaction mixture during lysis if a subsequent amplification step by PCR is contemplated (see for example Hedman and Rådström; PCR Detection of Microbial Pathogens: Second Edition, Methods in Molecular Biology, vol. 943, Chapter 2; Abu Al-Soud and Rådström; Journal of Clinical Microbiology, 2000, Vol. 38, No. 12, pp. 4463-4470).

These methods therefore have the drawbacks of being time consuming, tedious; of using chemical compounds that are often toxic for the operator and for the environment; of requiring expensive equipment, such as extractor hoods, protective gloves, centrifuges, etc.; and to be implemented in suitable environment contexts, such as laboratories.

However, there are many situations in which rapid extraction, not involving time consuming and laborious purification steps and not necessarily carried out in a laboratory setting, is advantageous. As a particularly illustrative example, this is the case of pandemic situations, wherein a great number of individuals need to be tested over short periods of time. Another example further concerns the case of the veterinarian wishing to quickly identify an infectious pathogen directly at the place where the sick animal is located. This so-called “point-of-care testing” (POCT) approach is the subject of special attention by the concerned professionals.

Moreover, it appears that depending on the nature of the original sample, the extraction protocols must often be adapted. For example, specific nucleic acid extraction kits have been developed on the market for blood samples (see for example the Qiagen® kit for nucleic acid extraction from a blood sample (QIAamp® DNA Mini and Blood Mini) or feces samples (QIAamp® Fast DNA Stool Mini)). One may note that these kits allow obtaining nucleic acids of a high degree of purity, compatible with most available analytical techniques, in approximately 20 to 30 minutes.

There is therefore a need to provide means allowing rapid extraction of nucleic acids, in an inexpensive manner, with little or no harmfulness for the manipulator and for the environment, and not requiring the constraint of an implementation in a dedicated laboratory.

There is also a need to provide means allowing “universal” nucleic acid extraction, regardless of the nature of the original sample containing the nucleic acids.

### SUMMARY

A first aspect of the invention relates to a kit for extracting nucleic acids contained in a complex sample, said kit comprising:
a lysis buffer comprising a concentration of SDS ranging from about 1% to about 25%, preferably from about 2.5% to about 20%;
a buffer comprising a concentration of a potassium salt ranging from about 0.1 M to about 5.0 M;
a buffer comprising a concentration of a zinc and/or copper salt ranging from about 0.5 M to about 5.0 M;
a filter having a pore diameter ranging from about 1 μm to about 10 μm, preferably from about 2 μm to about 7 μm;
optionally, an item chosen from the group comprising or consisting of one more syringe(s), one or more reaction tube(s), an instruction guide, and any combination thereof.

In some embodiments, the zinc salt is selected from a group comprising or consisting of zinc sulfate (ZnSO₄), and zinc chloride (ZnCl₂).

In certain embodiments, the copper salt is selected from a group comprising or consisting of copper sulfate (CuSO₄), and copper chloride (CuCl₂).

In some embodiments, the potassium salt is selected from the group comprising or consisting of potassium hydrogen carbonate (KHCO₃), potassium acetate (CH₃CO₂K), dipotassium hydrogen phosphate (K₂HPO₄), monobasic potassium phosphate (KH₂PO₄), and potassium chloride (KCl).

In certain embodiments, the lysis buffer and/or the buffer comprising a zinc and/or copper salt comprise(s) one or more additional compound(s) selected in the group comprising or consisting of Tris-HCl, HEPES, and MOPS.

One further aspect of the invention relates to the use of a kit according to the instant invention, in a method for extracting nucleic acids contained in a complex sample.

Another aspect of the invention pertains to a method for extracting nucleic acids contained in a complex sample, said method comprising the following steps:
a) contacting the complex sample with a lysis buffer comprising a concentration of SDS ranging from about 1% to about 25%, so that the final concentration of SDS in the reaction mixture is ranging from about 0.01% to about 10.0%;
b) optionally contacting the mixture from step a) with a buffer comprising a concentration of a zinc and/or copper salt ranging from about 0.5 M to about 5.0 M, so that the final salt concentration zinc and/or copper in the reaction mixture is ranging from about 10 mM to about 70 mM;
c) contacting the mixture of step a) or b) with a buffer comprising a concentration of a potassium salt ranging from about 0.1 M to about 5.0 M, so that the final concentration potassium salt in the reaction mixture is ranging from about 10 mM to about 500 mM;
d) filtering the reaction mixture from step c); and
e) collecting soluble nucleic acids.

In some embodiments, the complex sample is selected from the group comprising a biological sample, environmental sample, food sample, preferably a biological sample.

In certain embodiments, the biological sample is blood.

In some embodiments, step b) is performed when the complex sample is a blood sample.

In certain embodiments, the final concentration of SDS in the reaction mixture is ranging from about 0.01% to about 2%, preferably from about 0.01% to about 1.5%, preferably from about 0.01% to about 1%, preferably from about 0.25% to about 0.75%, when the complex sample is a blood sample.

In some embodiments, the final concentration of potassium salt in the reaction mixture is ranging from about 10 mM to about 500 mM, preferably from about 25 mM to about 150 mM, when the complex sample is a blood sample.

In certain embodiments, the biological sample is selected from the group consisting of feces sample, saliva sample, a product of respiratory lavage sample, and a nasopharyngeal secretion sample.

In some embodiments, the final concentration of SDS in the reaction mixture is ranging from about 0.06% to about 10%, preferably from about 0.1% to about 6.7%, when the complex sample is selected from group consisting of a feces sample, a saliva sample, a respiratory lavage sample, and a nasopharyngeal secretion sample.

In certain embodiments, the final concentration of potassium salt in the reaction mixture is ranging from about 60 mM to about 300 mM, preferably from about 100 mM to about 270 mM, when the complex sample is selected from the group consisting of a feces sample, a saliva sample, respiratory lavage sample, and a nasopharyngeal secretion sample.

### Definitions

In the present invention, the following terms have the following meanings:
“About” referring to a value, means more or less 10% of said value.
The term “comprise” is intended to mean “contain”, “encompass” and “include”. In some embodiments, the term “comprise” also encompasses the term “consist of”.
“Nucleic acids extraction” refers to the action of physically separating nucleic acids from other constituents of the biological entity that contains them. Since nucleic acids are contained in biological entities and protected from the environment by membranes and/or walls systems, in particular cell membranes and/or walls, the extraction of nucleic acids generally comprises a step of breaking or solubilizing the cell membranes and/or walls and releasing the nucleic acids into the reaction mixture; these phenomena are also defined as lysis.
“Sample” refers to a biological material obtained from an animal body, from an environment, or from a food product, preferably, obtained beforehand by an appropriate sampling technique on an individual or a product. By extension,
“Complex sample” relates to a sample which can comprise, in addition to the nucleic acids of interest, other constituents, such as polypeptides, lipids, polysaccharides, salts, metals, trace elements, solvents, acids, bases, and the likes.
“Buffer” refers to a solution that comprises a compound of interest (such as, e.g., SDS, a potassium salt). In some embodiments, the buffer also allows maintain its original pH, despite the limited addition of an acid or base or despite dilution.
“Reaction mixture” refers to an environment in which a reaction occurs. For example, biological entities contained in a sample and comprising nucleic acids of interest can be lysed upon being contacted with a buffer comprising SDS. Within the scope of the instant invention, the term “reaction mixture” is understood to mean the mixture of all the ingredients used by the extraction method of the invention, and comprises the sample containing the nucleic acids of interest, the lysis buffer, the buffer comprising the potassium salt and optionally the buffer comprising the zinc and/or copper salt.
“Amplification” refers to the specific multiplication of the number of copies of the nucleic acids of interest.
“Diagnosis” refers to both medical diagnosis and veterinary diagnosis and relates to the identification of the nature of the disease in a sick individual, in particular an infectious disease. According to some embodiments, the diagnostic methods described herein are non-invasive, i.e., are in vitro diagnostic methods. In these embodiments, a sample to be analyzed is obtained beforehand from the individual before the implementation of the methods of the invention.
“Individual” refers to an animal, vertebrate or invertebrate, preferably a mammal animal. The mammal animal may be a non-human mammal or a human.

### DETAILED DESCRIPTION

The inventors have observed that it is possible to extract nucleic acids contained in complex samples, and intended for analysis by amplification, in a quick and inexpensive manner, without additional purification steps being necessary. Furthermore, the method according to the invention does not require the use of compounds intended to trap amplification inhibitors which may be contained in complex samples. This method of extracting nucleic acids is thus compatible with the search for an infection by a pathogenic microorganism in a sick individual, and the ensuing diagnosis.

The present invention relates to a kit for extracting nucleic acids contained in a complex sample, said kit comprising:
a lysis buffer comprising a concentration of SDS ranging from about 1% to about 25%, preferably from about 2.5% to about 20%;
a buffer comprising a concentration of a potassium salt ranging from about 0.1 M to about 5.0 M;
a buffer comprising a concentration of a zinc and/or copper salt ranging from about 0.5 M to about 5.0 M;
a filter having a pore diameter ranging from about 1 μm to about 10 μm, preferably from about 2 μm to about 7 μm;
optionally, an item chosen from the group comprising or consisting of one or more syringe(s), one or more reaction tube(s), an instruction guide, and any combination thereof.

As used herein, and without otherwise statements, concentrations expressed as a percentage (%) refer to concentration as weight/volume (w/v), in particular as g/100 mL.

Illustratively, within the scope of the invention, an SDS concentration ranging from about 1% to about 25% is understood to refer to an SDS concentration ranging from about 1 g/100 mL to about and 25 g/100 mL.

In practice, the buffers of the kit are contained in dedicated receptacles, in particular vials or tubes. The receptacles can be of a suitable material, such as, e.g., polypropylene, acrylic, polyvinyl chloride.

In practice, the nucleic acids concerned by the invention are deoxyribonucleic acids (DNAs), ribonucleic acids (RNAs), including messenger ribonucleic acids (mRNAs), transfer ribonucleic acids (tRNAs), ribosomal ribonucleic acids (rRNAs).

In some embodiments, the nucleic acids are exclusively deoxyribonucleic acids (DNAs). In certain embodiments, the nucleic acids are exclusively ribonucleic acids (RNAs). In some embodiments, the nucleic acids are a mixture of deoxyribonucleic acids (DNAs) and ribonucleic acids (RNAs).

In certain embodiments, the nucleic acids are selected in the group comprising or consisting of a genomic nucleic acid and an extragenomic nucleic acid. Non-limitative examples of genomic nucleic acids include viral genomes and chromosomes, such as bacterial chromosomes, bacterial artificial chromosomes (BAC), eukaryotic chromosomes, and the likes. Non-limitative examples of extragenomic nucleic acids include plasmids, cosmids, viral vectors, and the likes.

In some embodiments, the nucleic acids are genomic nucleic acids.

In practice, the nature/origin of the sample may vary. Illustratively, the complex sample can be of biological, environmental, or food origin.

In certain embodiments, the sample is selected in the group comprising or consisting of a biological sample, an environmental sample and a food sample.

In some embodiments, the sample is a biological sample, particularly a biological fluid or a biopsy. In certain embodiments, the sample is a sample of a biological fluid, for example selected from the non-exhaustive list comprising, or consisting of, bile, feces, aqueous humor, milk, amniotic fluid, lymph, cerebrospinal fluid, plasma, product of respiratory lavage, product of a throat pouch, pus, nasopharyngeal secretion, lacrimal secretion, vaginal secretion, saliva, blood, serum, semen, and urine.

As used herein, a product of respiratory lavage includes a tracheal lavage, a bronchoalveolar lavage and a pulmonary lavage. In some embodiments, lavage and wash are intended to be equivalent terms.

In certain embodiments, the biological sample is selected in the group comprising or consisting of blood, feces, saliva, a product of respiratory lavage and a nasopharyngeal secretion.

In some embodiments, the sample is a biopsy, for example selected from the non-exhaustive list comprising, or consisting of, liver biopsy, intestinal biopsy, muscle biopsy, bone biopsy, pancreatic biopsy, skin biopsy, lung tissue biopsy, vascular tissue biopsy, bladder biopsy.

In some embodiments, the sample is an environmental sample such as seawater, freshwater, wastewater, ice, mud, soil, a surface.

As used herein, the term “waste water” refers to water which has been the subject of domestic, agricultural or industrial use, and circulates in a sewage network.

As used herein the term “surface” refers to the exterior or upper boundary of an object or body. In practice, any surface that may be contaminated with a biological entity may be encompassed by the invention. Within the scope of the invention, non-limitative examples of surfaces encompass, e.g., doorknob, light switch, table, chair, seat, floor, door, carpet, faucet handles, sink, kitchen countertops, toilet pan, ventilation exit, air purifier filter, clothes, wall, car wheel.

In some embodiments, the surface may be a domestic surface or a public area surface. Illustratively, public area surfaces may be encountered in educational institutions, shops, theatres, museums, restaurants, hospitals, ambulances, airports, ferry terminals, public transportation systems (subway, trains, buses), waiting rooms, and the likes.

In practice, surface samples may be collected by rubbing, wiping or swabbing according to good practice and protocols acknowledged in the state of the art. Illustratively, the rubbed, wiped or swabbed surface is at least about 1 cm², at least about 2 cm², at least about 5 cm², at least about 10 cm², at least about 20 cm², at least about 25 cm², at least about 50 cm², at least about 75 cm², or at least about 100 cm².

In some embodiments, the sample is a food sample, in particular, intended for animal or human consumption and containing an ingredient such as a cereal, e.g., wheat, barley, oats; a legume, e.g., lentils; meat, e.g., beef, veal, mutton, lamb, pork, rabbit, poultry; a fish, e.g., cod, trout, salmon; seafood, e.g., oysters, mussels, shrimp, lobster; a vegetable or a fruit, e.g., apricot, eggplant, banana, carrot, cherry, cabbage, zucchini, strawberry, green bean, kiwi, lettuce, pear, apple, potato. In some embodiments, the food sample can be from a manufactured food product, such as, e.g., a ready-made meal, a tin can, a frozen product.

In certain embodiments, the sample is a sample that may contain animal and/or plant cells.

In some embodiments, the animal cell is selected from the non-exhaustive list comprising an adipocyte; a fibroblast; an endothelial cell; an epithelial cell; a bone cell, e.g., an osteoblast, an osteocyte, an osteoclast; a muscle cell, e.g., a myoblast, a myocyte; a blood cell, e.g., a red blood cell, a lymphocyte, a polynuclear; a hepatocyte; a keratinocyte; a nerve cell; and any combination thereof.

In some embodiments, the sample is a sample which may contain a microorganism, such as an alga, an archaea, a bacterium, a fungus, a protozoan, a virus. In one embodiment, the microorganism is a pathogen for plants, for animals, and in particular for humans. In certain embodiments, the microorganism is referred to as a contaminant. In some embodiments, the sample comprises nucleic acids from one or more microorganism(s), in particular one or more contaminating microorganism(s), more particularly one or more pathogenic microorganism(s).

In certain embodiments, a bacterium pathogenic for an animal is selected from the non-exhaustive list comprising a bacterium of the species Afipia felis; a bacterium of the genus Anaplasma, preferably a bacterium of the species Anaplasma phagocytophilum, Anaplasma centrale, Anaplasma mesaeterum, Anaplasma platys, Anaplasma bovis, or Anaplasma ovis; a bacterium of the genus Bacillus, preferably a bacterium of the species Bacillus anthracis or Bacillus cereus; a bacterium of the genus Bartonella, preferably a bacterium of the species Bartonella henselae, or Bartonella clarridgeiae; a bacterium of the genus Bordetella, preferably a bacterium of the species Bordetella pertussis, Bordetella parapertussis or Bordetella bronchiseptica; a bacterium of the genus Borrelia, preferably a bacterium of the species Borrelia burgdorferi, Borrelia recurrentis, Borrelia hispanica, Borrelia persica, Borrelia duttonii or Borrelia crocidurae; a bacterium of the genus Brucella, preferably a bacterium of the species Brucella melitensis, Brucella abortus or Brucella suis; a bacterium of the genus Burkholderia, preferably a bacterium of the species Burkholderia mallei or Burkholderia pseudomallei; a bacterium of the genus Campylobacter, preferably a bacterium of the species Campylobacter fetus or Campylobacter jejuni; a bacterium of the genus Chlamydia, preferably a bacterium of the species Chlamydia trachomatis; a bacterium of the genus Chlamydophila, preferably a bacterium of the species Chlamydophila pneumoniae or Chlamydophila psittaci; a bacterium of the genus Clostridium, preferably a bacterium of the species Clostridium botulinum, Clostridium difficile, Clostridium perfringens or Clostridium tetani; a bacterium of the genus Corynebacterium, preferably a bacterium of the species Corynebacterium diphteriae; a bacterium of the genus Coxiella, preferably a bacterium of the species Coxiella burnetii; a bacterium of the genus Ehrlichia, preferably a bacterium of the species Ehrlichia chaffeensis, Ehrlichia equi or Ehrlichia phagocytophila; a bacterium of the genus Erysipelothrix, preferably a bacterium of the species Erysipelothrix rhusiopathiae; a bacterium of the genus Escherichia, preferably a bacterium of the species Escherichia coli; a bacterium of the genus Francisella, preferably a bacterium of the species Francisella tularensis; a bacterium of the genus Haemophilus, preferably a bacterium of the species Haemophilus ducreyi or Haemophilus influenzae; a bacterium of the genus Helicobacter, preferably a bacterium of the species Helicobacter pylori; a bacterium of the genus Legionella, preferably a bacterium of the species Legionella pneumophila; a bacterium of the genus Leptospira, preferably a bacterium of the species Leptospira interrogans; a bacterium of the genus Listeria, preferably a bacterium of the species Listeria monocytogenes; a bacterium of the genus Mycobacterium, preferably a bacterium of the species Mycobacterium leprae, Mycobacterium tuberculosis, Mycobacterium avium, Mycobacterium bovis or Mycobacterium intracellulare; a bacterium of the genus Mycoplasma, preferably a bacterium of the species Mycoplasma pneumoniae or Mycoplasma hominis; a bacterium of the genus Neisseria, preferably a bacterium of the species Neisseria gonorrhoeae or Neisseria meningitidis; a bacterium of the genus Pasteurella, preferably a bacterium of the species Pasteurella multocida; a bacterium of the genus Pseudomonas, preferably a bacterium of the species Pseudomonas aeruginosa; a bacterium of the genus Rickettsia; a bacterium of the genus Salmonella, preferably a bacterium of the species Salmonella enterica; a bacterium of the genus Shigella, preferably a bacterium of the species Shigella dysenteriae, Shigella flexneri, Shigella boydii or Shigella sonnei; a bacterium of the genus Spirillum, preferably a bacterium of the species Spirillum minus; a bacterium of the genus Staphylococcus, preferably a bacterium of the species Staphylococcus aureus; a bacterium of the genus Streptococcus, preferably a bacterium of the species Streptococcus pneumoniae; a bacterium of the genus Treponema, preferably a bacterium of the species Treponema pallidum; a bacterium of the genus Tropheryma, preferably a bacterium of the species Tropheryma whipplei; a bacterium of the genus Ureaplasma, preferably a bacterium of the species Ureaplasma urealyticum; a bacterium of the genus Vibrio, preferably a bacterium of the species Vibrio cholerae; a bacterium of the genus Yersinia, preferably a bacterium of the species Yersinia pestis, Yersinia enterocolitica, or Yersinia pseudotuberculosis.

In some embodiments, a fungus pathogenic for an animal is selected from the non-exhaustive list comprising, or consisting of, a fungus of the genus Aspergillus, a fungus of the genus Candida.

In certain embodiments, a protozoan pathogenic for an animal is selected from the non-exhaustive list comprising, or consisting of, a protozoan of the genus Babesia, a protozoan of the genus Plasmodium, a protozoan of the genus Theileria, a protozoan of the genus Toxoplasma.

In practice, the virus can be a DNA virus or an RNA virus.

In some embodiments, the DNA virus is selected from the non-exhaustive list comprising a virus of the family Adenoviridae, preferably a virus of the genus Adenovirus; of the Hepdnaviridae family, preferably a virus of the genus Herpadnavirus; from the family of Herpesviridae, preferably a virus of the genus Herpesvirus; of the Papovaviridae family, preferably a virus of the genus Polyomavirus or Papillomavirus; of the Parvoviridae family, preferably a virus of the Parvovirus genus; of the Poxviridae family, preferably a virus of the Poxvirus genus.

In certain embodiments, the DNA virus may be a double stranded (ds) DNA virus, in particular a virus selected in a group consisting of a virus of the Hepadnaviridae family, Circoviridae family, Herpesviridae family, Poxviridae family, Adenoviridae family, Papillomaviridae family, and Polyomaviridae family.

In some embodiments, the DNA virus may be a single stranded (ss) DNA virus, including a virus of the Parvoviridae family.

In certain embodiments, the RNA virus is selected from the non-exhaustive list comprising, or consisting of, a virus of the Arenaviridae family, preferably a virus of the Arenavirus genus; of the Bunyaviridae family, preferably a virus of the Bunyavirus, Phlebovirus, Nairovirus or Hantavirus genus; of the Caliciviridae family, preferably a virus of the Calicivirus genus; of the Coronaviridae family, preferably a virus of the Coronavirus genus; from the Filoviridae family, preferably a virus from the Filovirus genus; of the Flaviviridae family, preferably a virus of the Flavivirus, Ribivirus or Hepcivirus genus; from the Orthomyxoviridae family, preferably a virus of the genus Influenzavirus; of the Paramyxoviridae family, preferably a virus of the Pneumovirus, Paramyxovirus or Morbillivirus genus; of the Picornaviridae family, preferably a virus of the Enterovirus, Hepatovirus or Rhinovirus genus; of the Rhabdoviridae family, preferably a virus of the genus Lyssavirus or Vesiculovirus; of the Reoviridae family, preferably a virus of the Reovirus or Rotavirus genus; of the Retroviridae family, preferably a virus of the genus Oncornavirus or Lentivirus; from the Togaviridae family, preferably a virus from the genus Alphavirus.

In some embodiments, the RNA virus may be a double stranded RNA virus, including a virus of the Reoviridae family, in particular a rotavirus, such as, the bluetongue virus.

In certain embodiments, the RNA virus may be a negative-sense, single strand ss(−)RNA virus, in particular selected in a group consisting of a virus of the Orthomyxoviridae family, Filoviridae family, Paramyxoviridae family, Rhabdoviridae family, Arenaviridae family, Bunyaviridae family, Qinviridae family, Aspviridae family, and Yueviridae family.

In some embodiments, the RNA virus is a positive-sense, single strand ss(+)RNA virus, in particular selected in a group consisting of a virus of the Coronaviridae family, including SARS-CoV-2, SARS-CoV and MERS-CoV; a virus of the Picornaviridae family, including Poliovirus, Rhinovirus, Aphthovirus, Cardiovirus, Coxsackie Viruses, and Hepatitis A virus; a virus of the Calciviridae family, including Norwalk virus and Hepatitis E virus; a virus of the Flaviviridae family, including Yellow fever virus, West Nile Virus, Hepatitis C virus, Dengue virus and Zika virus; a virus of the Togaviridae family, including rubella virus and Chikungunya virus.

In certain embodiments, the virus of the Coronavirus genus may be a SARS-CoV-2 virus, a SARS-CoV virus, a MERS-CoV virus.

In some embodiments, the virus is selected from the non-exhaustive list comprising, or consisting of, African horse sickness virus, African swine fever virus, Andes virus, avian influenza virus, ovine bluetongue virus, Chapare virus, Chikungunya virus, Choclo virus, Crimean-Congo hemorrhagic fever virus, Dengue virus, Dobrava-Belgrade virus, Eastern equine encephalitis virus, Ebola virus, foot-and-mouth disease virus, goat pox virus, Guanarito virus, human immunodeficiency virus (HIV), Hantaan virus, Hendra virus, porcine herpesvirus (or Aujeszky's disease), swine fever virus, Japanese encephalitis virus, Junin virus, Kyasanur forest disease virus, Laguna negra virus, Lassa fever virus, ovine encephalomyelitis virus, Lujo virus, lumpy skin disease virus, Lymphocytic choriomeningitis virus, Machupo virus, Marburg virus, MERS-CoV virus, Monkey pox virus, Murray Valley encephalitis virus, Newcastle disease virus, Nipah virus, Omsk Haemorrhagic fever virus, Oropouche virus, small rodents plague virus, porcine type 9 enterovirus (or swine vesicular disease virus), Powassan encephalitis virus, rabies virus, Rift Valley virus, Rinderpest virus, Rocio virus, Sabia virus, SARS-CoV virus, SARS-CoV-2 virus, Seoul virus, Sheep pox virus, Sin Nombre virus, St. Louis encephalitis virus, Teschen's disease, tick-borne encephalitis virus, smallpox virus, Venezuelan equine encephalitis virus, vesicular stomatitis virus, western equine encephalitis virus, and yellow fever virus.

In practice, the sample can be of very varied origin.

In some embodiments, the sample is from an animal, preferably a mammal, preferably a human. In certain embodiments, the mammal is selected from the non-exhaustive list comprising, or consisting of, a cat, a horse, a goat, a dog, a guinea pig, a rabbit, a sheep, a pig, a rat, a mouse, a cow, a chicken. In some embodiments, the mammal is a pet selected from the non-exhaustive list comprising, or consisting of, a cat, a dog, a guinea pig, a rat, a mouse. In certain embodiments, the mammal is an animal of economic interest, for example selected from the non-exhaustive list comprising, or consisting of, a horse, a rabbit, a sheep, a pig, a cow, a poultry.

In certain embodiments, the biological, environmental or food samples are collected in compliance with good practices and possibly with the regulatory standards in force, in particular in human medicine and in veterinary medicine.

For example, the conditions of sterility may be desired. Samples may be collected using a suitable instrument, including but not limited to, syringe, toothpick, swab, spatula, receptacle, forceps, scalpel, adhesive paper.

In some embodiments, the blood samples are stored in suitable tubes, and containing for example heparin or EDTA, so as to prevent blood cells from clotting.

In certain embodiments, the sample is stored before carrying out the methods according to the present invention at a temperature ranging from about −196° C. to about 50° C., preferably from about −196° C. to about 25° C., from about −80° C. to about 25° C., from about −20° C. to about 25° C., from about 4° C. to about 25° C. In some embodiments, the sample is stored at ambient or room temperature before performing the method according to the present invention.

In some embodiments, the sample is stored before the implementation of the method according to the present invention for a period not exceeding about 7 days, preferably for a period not exceeding about 6 days, about 5 days, about 4 days, about 3 days, about 2 days, about 24 hours, about 12 hours, about 6 hours, about 2 hours, about 1 hour, about 30 minutes, about 15 minutes, about 10 minutes, about 2 minutes.

In practice, the lysis buffer comprises an SDS concentration ranging from about 1% to about 25%. Within the scope of the invention, the term “from about 1% to about 25%” includes about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% and 25%.

In some embodiments, the lysis buffer comprises an SDS concentration ranging from about 2.5% to about 20%. In certain embodiments, the lysis buffer comprises an SDS concentration ranging from about 5% to about 20%.

In practice, the final concentration of SDS in the reaction mixture is ranging from about 0.01% to about 10.0%. Within the scope of the invention, the term “from about 0.1% to about 10.0%” includes about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9% and 10.0%.

In some embodiments, the lysis buffer has a pH ranging from about 6.5 to about 9.0, preferably from about 7.0 to about 8.5, more preferably from about 7.5 to about 8.0. Within the scope of the invention, the term “ranging from about 6.5 to about 9.0” includes about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 and 9.0. In practice, the final pH of the lysis buffer, i.e., a pH ranging from about 6.5 to about 9.0, may be obtained by the addition of an acid or a base into a solution comprising SDS.

Within the scope of the invention, the lysis buffer is more particularly intended to promote the lysis of the membrane or wall systems of biological entities, such as, e.g., cells, viruses, containing the nucleic acids of interest.

In practice, the concentration of the buffer comprising a zinc and/or copper salt is ranging from about 0.5 M to about 5.0 M. Within the scope of the invention, the term “ranging from about 0.5 M to about 5.0 M” includes about 0.5 M, 0.6 M, 0.7 M, 0.8 M, 0.9 M, 1.0 M, 1.2 M, 1.4 M, 1.6 M, 1.8 M, 2.0 M, 2.2 M, 2.4 M, 2.6 M, 2.8 M, 3.0 M, 3.2 M, 3.4 M, 3.6 M, 3.8 M, 4.0 M, 4.2 M, 4.4 M, 4.6 M, 4.8 M and 5.0 M.

In certain embodiments, the buffer comprises a zinc salt. In practice, the buffer may not comprise copper. In some embodiments, the buffer comprises a copper salt. In practice, the buffer may not comprise zinc. In certain embodiments, the buffer comprises a zinc salt or a copper salt. In certain embodiments, the buffer comprises a zinc salt and a copper salt.

In practice, the final concentration of zinc and/or copper salt in the reaction mixture is ranging from about 10 mM to about 70 mM. As used herein, the term “from about 10 mM to about 70 mM” includes about 10 mM, 15 mM, 20 mM, 25 mM, 30 mM, 35 mM, 40 mM, 45 mM, 50 mM, 55 mM, 60 mM, 65 mM, and 70 mM.

Within the scope of the invention, the buffer comprising the zinc and/or copper salt is particularly intended for precipitating hemoglobin contained in blood samples or certain samples containing blood, such as for example biopsies. Indeed, hemoglobin is known to be incompatible with certain steps of nucleic acid analysis used in many methods, such as amplification, in particular PCR amplification.

In practice, the zinc salt may advantageously be chosen from a group comprising or consisting of zinc sulfate (ZnSO₄), and zinc chloride (ZnCl₂).

In practice, the copper salt may advantageously be chosen from a group comprising or consisting of copper sulfate (CuSO₄), and copper chloride (CuCl₂).

These salts can be prepared extratemporaneously or alternatively be acquired commercially.

In one embodiment, the zinc salt is zinc sulfate (ZnSO₄). In one embodiment, the copper salt is copper sulfate (CuSO₄).

In some embodiments, the buffer comprising the zinc and/or copper salt has a pH ranging from about 6.5 to about 9.0, preferably from about 7.0 to about 8.5, more preferably from about 7.5 to about 8.0. In practice, the final pH of the buffer comprising the zinc and/or copper salt, i.e., a pH ranging from about 6.5 to about 9.0, may be obtained by the addition of an acid or a base into a solution comprising a zinc and/or copper salt.

In practice, the concentration of the buffer comprising a potassium salt is ranging from about 0.1 M to about 5.0 M. Within the scope of the invention, the term “from about 0.1 M to about 5.0 M” includes about 0.1 M, 0.2 M, 0.3 M, 0.4 M, 0.5 M, 0.6 M, 0.7 M, 0.8 M, 0.9 M, 1.0 M, 1.2 M, 1.4 M, 1.6 M, 1.8 M, 2.0 M, 2.2 M, 2.4 M, 2.6 M, 2.8 M, 3.0 M, 3.2 M, 3.4 M, 3.6 M, 3.8 M, 4.0 M, 4.2 M, 4.4 M, 4.6 M, 4.8 M and 5.0 M.

In practice, the final concentration of the buffer comprising a potassium salt in the reaction mixture is ranging from about 10 mM to about 500 mM. Within the scope of the invention, the term “from about 10 mM to about 500 mM” includes about 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 310 mM, 320 mM, 330 mM, 340 mM, 350 mM, 360 mM, 370 mM, 380 mM, 390 mM, 400 mM, 410 mM, 420 mM, 430 mM, 440 mM, 450 mM, 460 mM, 470 mM, 480 mM, 490 mM and 500 mM.

The buffer comprising the potassium salt is intended to precipitate SDS which is incompatible with nucleic acid analysis steps carried out in many methods, such as, for example, amplification, in particular amplification by PCR.

In practice, the potassium salt can be advantageously chosen from the group comprising or consisting of potassium hydrogen carbonate (KHCO₃), potassium acetate (CH₃CO₂K), dipotassium hydrogen phosphate (K₂HPO₄), monobasic potassium phosphate (KH₂PO₄), and potassium chloride (KCl).

These salts can be prepared extratemporaneously or alternatively be acquired commercially.

In some embodiments, the potassium salt is potassium hydrogen carbonate (KHCO₃).

In practice, the filter has a pore diameter ranging from about 1 μm to about 10 μm. Within the scope of the invention, the term “from about 1 μm to about 10 μm” includes about 1 μm, 8 μm, 9 μm and 10 μm.

In certain embodiments, the filter has a membrane having pores with a diameter ranging from about 2 μm to about 7 μm, in particular a diameter of about 5 μm.

The filter is more particularly intended to retain the precipitate which contains the SDS, the potassium salt, and possibly the insoluble cellular debris. When the sample is a blood sample or a sample containing blood, the filter is also intended to retain the precipitate which contains hemoglobin and zinc and/or copper. On the contrary, the filter is rather intended to allow the passing through of the nucleic acids contained in the sample. In practice, both genomic and extragenomic nucleic acids are allowed to pass through the filter, and are not therefore retained by the filter.

In some embodiments, the membrane of the filter is made of cellulose acetate, cellulose nitrate, nylon, polyamide, silica. Filters suitable for the implementation of the invention can be purchased commercially, for example from Sartorius®, Hahnemühle®, Sebio®, or Whatman™.

In certain embodiments, at least one of the buffers of the kit, i.e., the lysis buffer, the buffer containing a potassium salt, or the buffer containing a zinc and/or copper salt, comprises an RNase inhibitor. These embodiments are particularly advantageous when the nucleic acids of interest contained in the sample are ribonucleic acids (RNAs).

In some embodiments, the lysis buffer and the buffer comprising a zinc and/or copper salt may be combined as a unique buffer. In practice, the buffer comprising a zinc and/or copper salt may not be combined with the buffer comprising a potassium salt as a unique buffer, because zinc may precipitate in the presence of bicarbonate.

In certain embodiments, the lysis buffer and/or the buffer comprising a zinc and/or copper salt may further comprise one or more additional compound(s). In some embodiments, the lysis buffer and/or the buffer comprising a zinc and/or copper salt comprise(s) one or more additional compound(s) selected in the group comprising or consisting of Tris-HCl, HEPES, and MOPS.

In some embodiments, the concentration of Tris-HCl, HEPES, or MOPS in the lysis buffer and/or the buffer comprising a zinc and/or copper salt ranges from about 0.5 M to about 1.5 M. Within the scope of the invention, the term “from about 0.5 M to about 1.5 M” includes 0.5 M, 0.6 M, 0.7 M, 0.8 M, 0.9 M, 1.0 M, 1.1 M, 1.2 M, 1.3 M, 1.4 M and 1.5 M.

In practice, the final concentration of Tris-HCl, HEPES, or MOPS in the lysis buffer and/or the buffer comprising a zinc and/or copper salt ranges from about 1 mM to about 100 mM. Within the scope of the invention, the term “from about 1 mM to about 100 mM” includes 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM and 100 mM.

In certain embodiments, the one or more syringe(s) is/are intended to be used with the filter.

The invention also relates to the use of a kit according to the invention in a method for extracting nucleic acids contained in a complex sample.

The methods according to the invention may be carried out in vitro or ex vivo.

The invention relates to a method for extracting nucleic acids contained in a complex sample, said method using the kit according to the invention.

The invention also relates to a method for extracting nucleic acids contained in a complex sample, said method comprising the following steps:
a) contacting the complex sample with a lysis buffer comprising a concentration of SDS ranging from about 1% to about 25%, so that the final concentration of SDS in the reaction mixture is ranging from about 0.01% to about 10.0%;
b) optionally contacting the mixture from step a) with a buffer comprising a concentration of a zinc and/or copper salt ranging from about 0.5 M to about 5.0 M, so that the final salt concentration zinc and/or copper in the reaction mixture is ranging from about 10 mM to about 70 mM;
c) contacting the mixture of step a) orb) with a buffer comprising a concentration of a potassium salt ranging from about 0.1 M to about 5.0 M, so that the final concentration potassium salt in the reaction mixture is ranging from about 10 mM to about 500 mM;
d) filtering the reaction mixture from step c), so as to collect soluble nucleic acids.

The invention further relates to a method for extracting nucleic acids contained in a complex sample, said method comprising the following steps:
a) contacting the complex sample with a lysis buffer comprising a concentration of SDS ranging from about 1% to about 25%, so that the final concentration of SDS in the reaction mixture is ranging from about 0.01% to about 10.0%;
b) optionally contacting the mixture from step a) with a buffer comprising a concentration of a zinc and/or copper salt ranging from about 0.5 M to about 5.0 M, so that the final salt concentration zinc and/or copper in the reaction mixture is ranging from about 10 mM to about 70 mM;
c) contacting the mixture of step a) orb) with a buffer comprising a concentration of a potassium salt ranging from about 0.1 M to about 5.0 M, so that the final concentration potassium salt in the reaction mixture is ranging from about 10 mM to about 500 mM;
d) filtering the reaction mixture from step c); and
e) collecting soluble nucleic acids.

In some embodiments, the method according to the invention consists of steps a) to e) above-mentioned. In certain embodiments, the method according to the invention does not comprise any further purification step, such as e.g., a step involving a purification column, phenol and/or chloroform, centrifugation, and the likes.

Within the scope of the invention, the complex sample in step a) comprises at least 1 copy of a nucleic acid, preferably at least 10 copies of a nucleic acid, preferably at least 25 copies of a nucleic acid to be extracted.

In some embodiments, the method is implemented by means of a kit according to the invention.

As used herein, “reaction mixture” is meant to refer to the mixture of all the ingredients at the end of step c). Thus, the “reaction mixture” within the meaning of the invention comprises the sample containing the nucleic acids of interest, the lysis buffer, the buffer comprising the potassium salt and optionally the buffer comprising the zinc and/or copper salt.

In practice, the final concentration of SDS in the reaction mixture is ranging from about 0.01% to about 10.0%. Within the scope of the invention, the term “from about 0.1% to about 10.0%” includes about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2%, 4.4%, 4.6%, 4.8%, 5.0%, 5.2%, 5.4%, 5.6%, 5.8%, 6.0%, 6.2%, 6.4%, 6.6%, 6.8%, 7.0%, 7.2%, 7.4%, 7.6%, 7.8%, 8.0%, 8.2%, 8.4%, 8.8%, 9.0%, 9.2%, 9.4%, 9.6%, 9.8% and 10.0%.

In practice, the final concentration of zinc and/or copper salt in the reaction mixture is ranging from about 10 mM to about 70 mM. As used herein, the term “from about 10 mM to about 70 mM” includes about 10 mM, 20 mM, 30 mM, 40 mM, 50 mM, 60 mM and 70 mM.

In practice, the final concentration of potassium salt in the reaction mixture is ranging from about 10 mM to about 500 mM. Within the scope of the invention, the term “from about 10 mM to about 500 mM” includes about 10 mM, 20 mM, 30 mM, 40 mM, 50 mM, 60 mM, 70 mM, 80 mM, 90 mM, 100 mM, 120 mM, 140 mM, 160 mM, 180 mM, 200 mM, 220 mM, 240 mM, 260 mM, 280 mM, 300 mM, 320 mM, 340 mM, 360 mM, 380 mM, 400 mM, 420 mM, 440 mM, 460 mM, 480 mM and 500 mM.

In certain embodiments, the complex sample is selected from the group comprising a biological sample, environmental sample, food sample, preferably a biological sample.

In some embodiments, the sample, in particular the biological sample, is a biological fluid sample. In some embodiments, the biological fluid is selected from the group consisting of bile, feces, aqueous humor, milk, amniotic fluid, lymph, cerebrospinal fluid, plasma, the product of respiratory lavage, the product of a throat pouch, pus, nasopharyngeal secretion, lacrimal secretion, vaginal secretion, saliva, blood, serum, semen, and urine. In certain embodiments, the biological fluid is selected from the group comprising feces, saliva, nasopharyngeal secretion, blood.

In some embodiments, the biological sample, in particular the biological fluid, is blood.

In certain embodiments, step b) is performed when the complex sample is a blood sample.

In some embodiments, the final concentration of SDS in the reaction mixture is ranging from about 0.01% to about 2%, preferably from about 0.01% to about 1.5%, preferably from about 0.01% to about 1%, preferably from about 0.25% to about 0.75%, when the complex sample is a blood sample.

Within the scope of the invention, the term “from about 0.01% to about 2%” includes about 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.2%, 1.3%, 1.4%, 1.5%, 1.6%, 1.7%, 1.8%, 1.9% and 2%.

In certain embodiments, the final concentration of potassium salt in the reaction mixture is ranging from about 10 mM to about 500 mM, preferably from about 25 mM to about 150 mM, when the complex sample is a blood sample.

In certain embodiments, the final concentration of potassium salt in the reaction mixture is ranging from about 15 mM to about 300 mM, preferably from about 20 mM to about 250 mM, when the complex sample is a blood sample.

In some embodiments, the biological sample, in particular the biological fluid, is selected from the group consisting of feces sample, saliva sample, a product of respiratory lavage sample, and a nasopharyngeal secretion sample.

In certain embodiment, the final concentration of SDS in the reaction mixture is ranging from about 0.06% to about 10%, preferably from about 0.1% to about 6.7%, when the complex sample is selected from group consisting of a feces sample, a saliva sample, a respiratory lavage sample, and a nasopharyngeal secretion sample.

Within the scope of the invention, the term “from about 0.06% to about 10%” includes about 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, 5%, 5.2%, 5.4%, 5.6%, 5.8%, 6%, 6.2%, 6.4%, 6.6%, 6.8%, 7%, 7.2%, 7.4%, 7.6%, 7.8%, 8%, 8.2%, 8.4%, 8.6%, 8.8%, 9%, 9.2%, 9.4%, 9.6%, 9.8% and 10%.

In some embodiments, the final concentration of potassium salt in the reaction mixture is ranging from about 60 mM to about 300 mM, preferably from about 100 mM to about 270 mM, when the complex sample is selected from the group consisting of a feces sample, a saliva sample, respiratory lavage sample, and a nasopharyngeal secretion sample.

Within the scope of the invention, the term “from about 60 mM to about 300 mM” includes about 60 mM, 80 mM, 100 mM, 120 mM, 140 mM, 160 mM, 180 mM, 200 mM, 220 mM, 240 mM, 260 mM, 280 mM and 300 mM.

In certain embodiments, step a) and/or optionally step b) is/are performed under neutral pH.

As used herein, the term “neutral pH” includes a pH ranging from about 6.5 to about 9.0, preferably from about 7.0 to about 8.5, preferably from about 7.5 to about 8.0. In these embodiments, the lysis is not performed in alkaline conditions (i.e., at basic pH), hence is not an alkaline lysis.

In some embodiments, step a) and/or optionally step b) is/are performed under acidic pH. As used herein, the term “acidic pH” refers to a pH below 6.5. In some alternative embodiments, step a) and/or optionally step b) is/are performed under basic pH. As used herein, the term “basic pH” refers to a pH above 9.0.

In certain embodiments, the mixture at the end of step a), optionally of step b) and of step c) is homogenized, preferably by gentle stirring, more preferably by inversion of the tube.

In practice, the implementation of the method according to the invention allows obtaining a solution of nucleic acids in less than about 5 min, preferably in less than about 3 min, preferably in less than about 2 min.

In practice, the method according to the invention may be carried out at ambient or room temperature. Within the scope of the invention, the expression “ambient temperature” includes a temperature ranging from about −5° C. to about 40° C., i.e., temperatures of about −5° C., −4° C., −3° C., −2° C., −1° C., 0° C., 1° C., 2° C., 3° C., 4° C., 5° C., 6° C., 7° C., 8° C., 9° C., 10° C., 12° C., 14° C., 16° C., 18° C., 20° C., 22° C., 24° C., 26° C., 28° C., 30° C., 32° C., 34° C., 36° C., 38° C. and 40° C. In some embodiments, the temperature is ranging from about 5° C. to about 40° C.

The kit and the method according to the invention may be implemented in various circumstances:
identifying the presence of a potentially pathogenic microorganism in a biological, environmental or food sample;
performing a metagenomic analysis, i.e., analyzing the composition of living organisms in a sample;
identifying one or more biomarker(s) associated with a pathology in a biological sample.

In practice, the method according to the invention may be preferred when access to laboratory equipment is difficult, or when the urgency of the analysis of the nucleic acids of interest requires rapid action, such as, e.g., during a pandemic situation.

The nucleic acids extracted by a kit or a method according to the invention may be subsequently analyzed to identify the presence of a potentially pathogenic microorganism and thus diagnose an infectious disease.

In certain embodiments, the extraction of nucleic acids according to the invention may be implemented for the diagnosis of a disease, in particular a disease in a human individual, selected from the group comprising SARS-CoV-2 viral infection, smallpox, flu (Influenza), measles, mumps, rubella, chicken pox, shingles, hepatitis, herpes, polio, rabies, Lyme disease, pneumonia, plague, Ebola, HIV, tuberculosis, typhus, severe acute respiratory syndrome (SARS), Dengue fever, Zika, yellow fever, and Epstein-Barr.

In certain embodiments, the extraction of nucleic acids according to the invention may be implemented for the diagnosis of a canine disease selected from the group comprising an anaplasmosis (bacteria of the genus Anaplasma), canine coronavirosis, eczema of parasitic origin, canine ehrlichiosis (bacteria of the species Ehrlichia canis), scabies (mites: Otodecte cynotis, Sarcoptes scabiei, Dermodex canis), gastroenteritis, giardiasis (parasite of the genus Giardia), hepatitis (adenovirus CAV1), canine hemobartonellosis, leishmaniasis (Leshmania Infantum), leptospirosis (bacteria of the genus Leptospira), distemper (virus of the genus Morbillivirus), Lyme disease or borreliosis (bacteria of the species Borrelia Burgdorferi), otitis, parvovirosis, piroplasmosis or babesiosis (Babesia canis parasite), rabies (Lyssavirus rabies virus), spirocerciasis (Spirocerca Lupi parasite), ringworm (fungus Microsporum canis, Trichophyton mentagrophytes, Microsporum gypseum), a tapeworm (Taenia worm, Dipylidium, Echinococcus), a tetanus (Clostridium tetani bacterium), kennel cough (association of canine Parainfluenza virus or an adenovirus with the bacterium Bordetella Bronchiseptica or Pseudomonas aeruginosa) and tuberculosis (Mycobacterium bovis or Mycobacterium tuberculosis bacteria).

In certain embodiments, the extraction of nucleic acids according to the invention may be implemented for the diagnosis of a feline disease selected from the group comprising an anaplasmosis, a bordetellosis, a borreliosis, a calicivirosis, a feline chlamydophilosis, conjunctivitis, chlamydia, coronavirosis, echinococcosis, encephalitis, giardiasis, gingivitis, herpesvirosis, hemobartonellosis, auricular mange or otacariosis or otitis, feline leukosis (FeLV), feline piroplasmosis, feline viral rhinotracheitis (FVR), rickettsiosis, cat AIDS (FIV), feline urologic syndrome (FUS), ringworm, toxoplasmosis, feline tritrichomoniasis, tuberculosis, and typhus.

In some embodiments, the extraction of nucleic acids according to the invention may be implemented for the diagnosis of an equine disease selected from the group comprising anaplasmosis, babesiosis, ehrlichiosis, equine influenza, strangles, leptospirosis, Lyme disease, piroplasmosis, rhinopneumonia, theileriosis.

The nucleic acids extracted by the means of the kit or the method according to the invention may be analyzed by any suitable technique described in the state of the art, or a technique derived therefrom. Illustratively, a technique suitable for analysis of a nucleic acid may involve an amplification reaction, such as a thermal amplification reaction. Examples of thermal amplification reactions include, but are not limited to, the polymerase chain reaction (or PCR) and its variants, e.g., Hot-start PCR, Touchdown PCR, Nested PCR, Fast PCR, Direct PCR, Multiplex PCR, Long PCR, GC-rich PCR, Inverse PCR, Quantitative PCR, Ligation-Mediated PCR, Multiplex ligation-dependent probe amplification PCR, Methylation-specific PCR, Anchored PCR, Asymmetric PCR, Thermal Asymmetric Interlaced PCR, Assembly PCR, Allele-Specific PCR, Arbitrarily Primed PCR.

In practice, the amplification reaction may be an isothermal amplification reaction. Examples of isothermal amplification reactions include, but are not limited to, isothermal loop amplification (or ‘Loop Mediated Isothermal Amplification’, LAMP), Recombinase Polymerase Amplification (or RPA), Nucleic Acid Sequence-Based Amplification (or NASBA), Helicase-Dependent Amplification (or HDA), circular amplification (or ‘Rolling Circle Amplification’, RCA), Strand Displacement Amplification (SDA) and Multiple Displacement Amplification (or MDA).

In practice, the amplification reaction is an isothermal amplification reaction of the isothermal loop amplification type (“Loop Mediated Isothermal Amplification”, LAMP), in particular as described by Notomi et al. (Nucleic Acids Res., 2000, 28 (12): e63), by patent No. EP1020534, or by Nagamine et al. (Mol Cell Probes., 2002, 16 (3): 223-9).

The LAMP amplification reaction uses a strand displacement polymerase, typically a Bst polymerase (extracted from the bacterium Bacillus stearothermophilus), Aac polymerase, phi-29 Vent polymerase, or a variant of these polymerases. The LAMP amplification reaction may also be performed directly for the amplification of RNA to DNA, by adding reverse transcriptase to the reaction mixture. An example of a reverse transcriptase includes, without limitation, AMV reverse transcriptase. In some embodiments, the polymerase has both DNA- and RNA-dependent DNA polymerase activity, and the amplification reaction does not require the addition of a reverse transcriptase.

Kits allowing the implementation of the LAMP amplification reaction are commercially available and accessible to those skilled in the art.

Illustratively, the LAMP amplification reaction may be carried out at a constant temperature of about 50° C., 55° C., 60° C., 60.5° C., 61° C., 61.5° C., 62° C., 62.5° C., 63° C., 63.5° C., 64° C., 64.5° C., 65° C., 65.5° C., 66° C., 66.5° C., 67° C., 67.5° C., 68° C., 68.5° C., 69° C., 69.5° C., 69.5° C., 70° C., 75° C. or more.

Illustratively, the LAMP amplification reaction may be carried out for a period of at least about 2 minutes, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes or more. In some embodiments, the LAMP amplification reaction may be carried out for a period ranging from about 10 minutes to about 60 minutes, preferably from about 15 minutes to about 45 minutes.

Illustratively, the LAMP amplification reaction may be carried out using an isothermal amplification system. Several such instruments are available on the market, and accessible to those skilled in the art.

Illustratively, the reaction for amplifying the nucleic acids extracted according to the methods of the present invention comprises a step of detecting the amplicon, i.e., a step of detecting the product of the amplification of the target nucleotide sequence.

The techniques for detecting an amplicon are well known to those skilled in the art, and include, without being limited thereto, the use of DNA intercalating agents (e.g., ethidium bromide, propidium iodide, SYBR green, SYBR gold, crystal violet, GelRed, DAPI); the measurement of colorimetry and/or fluorescence; the use of oligonucleotide probes labeled by fluorescence; the presence of an antigen or radioactivity; the use of dNTPs labeled with fluorescence or radioactivity; enzymatic assays; analysis of change in absorbance; turbidimetry; and lateral flow assays.

In practice, the amplicon detection step involves the use of DNA intercalating agents. According to this embodiment, detection of the amplicon may be performed with the naked eye, by observing the colorimetry of the sample, or by measuring the fluorescence emitted by the intercalating agent. For example, the addition of an intercalating agent of the SYBR green type provides an orange color to the sample in the absence of intercalation (i.e., in the absence of amplification of a target sequence) or green/blue color in the presence intercalation (i.e., in the presence of amplification of a target sequence) (see e.g., Iwamoto et al; J Clin Microbiol., 2003, 41 (6): 2616-22). This type of intercalating agent may also be detected by fluorescence, which is emitted only when the agent intercalates between the nucleotide bases of a double strand of DNA (i.e., in the presence of amplification of a target sequence).

Alternatively, the amplicon detection step may involve the use of fluorescent-labeled oligonucleotide probes and may include the detection and/or measurement of fluorescence. According to this embodiment, the oligonucleotide probes are able to hybridize to the amplicon.

In certain embodiments, both the extraction and the analysis of nucleic acids comprised in a complex sample, as disclosed herein, may be performed from about 15 min to about 60 min, preferably from about 20 to about 45 min.

### BRIEF DESCRIPTION OF THE DRAWINGS

FIG. 1 is a diagram showing the protocol for extracting nucleic acids from a complex sample, according to the invention. From left to right, all or part of a sample is contacted with a lysis buffer containing SDS, thus forming a first reaction mixture (step 1). For blood samples or samples containing blood, the addition of zinc and/or copper, in the first reaction mixture, allows hemoglobin to be precipitated. The SDS contained in the first reaction mixture is then precipitated by means of a buffer containing a potassium salt, which is introduced into the first reaction mixture (step 2). The second reaction mixture following the step of precipitating SDS with potassium is then filtered using a suitable filter (step 3). The SDS/potassium precipitate is retained by the filter, as is the hemoglobin/zinc and/or copper precipitate, when the sample is a blood sample or contains blood. The nucleic acids contained in the second reaction mixture and from the sample are not retained by the filter and can be collected in a suitable receptacle, for example a tube. Nucleic acids extracted from the sample can be analyzed in a later step (step 4).

### EXAMPLES

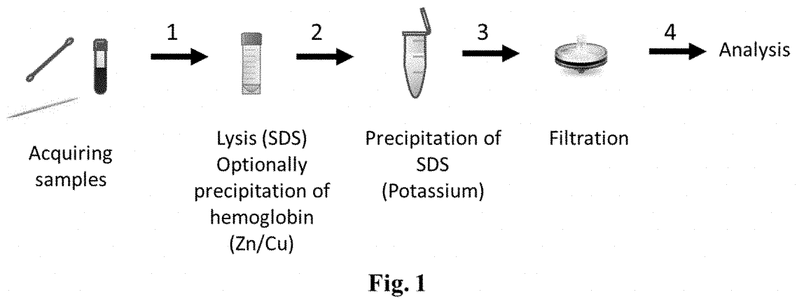

The present invention is further illustrated by the following non-limitative examples and by FIG. 1.

### Example 1: Extraction of Nucleic Acids from a Sample of Cat Feces

### 1.1 Materials and Methods

A sample of cat feces is collected with a sterile toothpick. The fecal matter contained on the toothpick is placed in a tube containing 2 mL of lysis buffer containing SDS. The toothpick is shaken for a few seconds so as to move the fecal matter from the toothpick to the lysis buffer. The toothpick is then removed. The tube containing the reaction mixture is then shaken, so as to homogenize the fecal matter with the buffer. 1 mL of a buffer containing KHCO₃ is then added to the reaction mixture, which is gently stirred by inverting the tube 3 or 4 times. The reaction mixture has a final volume of 3 mL. The contents of the tube are then poured into a 5 mL syringe at the end of which is a cellulose acetate filter with a pore diameter of approximately 5 μm (Sartorius®). Filtration is carried out so that the filtrate is collected in a sterile tube. Nucleic acids are analyzed as follows. The presence of the feline domestic gene encoding the subunit 5 of NADH dehydrogenase (NDSS) (GenBank accession number: NC_001700.1) is determined after isothermal amplification at 65° C. for 30 min.

### 1.2 Results

TABLE 1
Determination of the effective concentrations of SDS and KHCO₃ for the
extraction of nucleic acids from a sample of cat feces.

| SDS conc. (%)¹ | Amplification² | KHCO₃ conc. (mM)¹ | Amplification² |
|---|---|---|---|
| 0.067% | +(2/2) | 67 mM | +(2/2) |
| 0.67% | +(3/3) | 133 mM | +(5/5) |
| 1.3% | +(3/3) | 267 mM | +(2/5) |
| 3.33% | +(2/2) | 533 mM | −(1/1) |

| | |
|---|---|
| 6.67% | +(1/2) |
| 13.3% | −(2/2) |

¹Final concentration in the final reaction mixture comprising the feces, the buffer comprising the SDS and the buffer comprising the KHCO₃ (total final volume of the reaction mixture before filtration: 3 mL).
²Number of assays in which specific amplification of the domestic gene is observed over the total number of assays.

In conclusion, the final concentration of SDS in the reaction mixture is between approximately 0.06% and 10%, and that of KHCO₃ is between approximately 60 mM and 300 mM, for an extraction of nucleic acids from an animal's feces sample.

### Example 2: Determination of the Effective SDS Concentration to Ensure the Extraction of Nucleic Acids from a Blood Sample

The freshly drawn blood samples are immediately contacted with a buffer comprising 20% SDS. The final SDS concentrations in the reaction mixture are between 0.33% and 2.5%, i.e., 0.33%, 1.67%, 2.5%. The final SDS concentration of 0.33% results in a clear lysate. In contrast, the final SDS concentrations of 1.67% and 2.5% lead to clotting of blood cells.

In conclusion, the final concentration of SDS in the reaction mixture should not be greater than 1%, for extraction of nucleic acids from an animal blood sample.

### Example 3: Determination of the Effective Concentrations of ZnSO₄ and KHCO₃ for the Extraction of Nucleic Acids from a Sample of Horse Blood

### 3.1 Materials and Methods

A horse blood sample is collected according to the usual practices. To 0.6 mL of collected blood are added 30 μL of a lysis buffer containing 20% SDS and a volume of 30 μL of a buffer containing ZnSO₄. The tube containing the reaction mixture is then gently stirred by inverting the tube 3 or 4 times, so as to homogenize. 1.14 mL of a buffer containing KHCO₃ are then added to the reaction mixture, which is gently stirred by inverting the tube 3 or 4 times. When the ZnSO₄ concentration varies, the final KHCO₃ concentration is set at 100 mM. When the KHCO₃ concentration varies, the final ZnSO₄ concentration is set at 33 mM. The reaction mixture (final volume 1.8 mL) is filtered as described in Example 1. The presence of the equine domestic gene encoding (32 microglobulin (B2M gene) (GenBank accession number: AH011712.2) is determined after an isothermal amplification at 65° C. for 30 min.

### 3.2 Results

TABLE 2
Determination of the effective ZnSO₄ and KHCO₃ concentrations for the
extraction of nucleic acids from a horse blood sample

| ZnSO₄ conc. (mM)¹ | Amplification² | KHCO₃ conc. (mM)¹ | Amplification² |
|---|---|---|---|
| 8 mM | +(3/4) | 0 mM | −(1/1) |
| 10 mM | +(1/1) | 8 mM | +(1/1) |
| 17 mM | +(1/1) | 17 mM | +(1/1) |
| 25 mM | +(1/2) | 25 mM | +(2/2) |
| 30 mM | +(1/4) | 32 mM | +(1/1) |
| 32 mM | +(2/2) | 37 mM | +(1/1) |
| 33 mM | +(11/12) | 40 mM | +(1/1) |
| 40 mM | −(2/2) | 47.5 mM | +(1/1) |
| 50 mM | −(3/3) | 50 mM | +(1/1) |
| 70 mM | −(3/3) | 100 mM | +(11/12) |
| | | 116 mM | +(6/10) |
| | | 133 mM | +(1/2) |

¹Final concentration in the final reaction mixture comprising blood, the buffer comprising SDS, the buffer comprising ZnSO₄ and the buffer comprising KHCO₃ (total final volume before filtration: 1.8 mL).
²Number of assays in which specific amplification of the domestic gene is observed over the total number of assays.

In conclusion, the final concentration of ZnSO₄ in the reaction mixture is between about 8 mM and 40 mM, and that of KHCO₃ is greater than 8 mM, for extraction of nucleic acids from a horse blood sample.

### Example 4: Extraction of Nucleic Acids from a Cat Blood Sample

A cat blood sample is collected according to veterinary practice. To 1 mL of collected blood are added 50 μL of a lysis buffer containing 20% SDS and 50 μL of a buffer containing ZnSO₄. The tube containing the reaction mixture is then gently stirred by inverting the tube 3 or 4 times, so as to homogenize. 2 mL of a buffer containing KHCO₃ are then added to the reaction mixture, which is gently stirred by inverting the tube 3 or 4 times. The reaction mixture is then filtered as in Example 1 or 3, and the presence of the feline domestic gene encoding ND5S is shown after isothermal amplification at 65° C. for 30 min.

### Example 5: Extraction of Nucleic Acids from a Dog Blood Sample

### 5.1 Materials and Methods

A dog blood sample is collected according to veterinary practice, and treated as described in Example 4. When the ZnSO₄ concentration varies, the final KHCO₃ concentration is set at 40 mM. When the KHCO₃ concentration varies, the final ZnSO₄ concentration is set at 33 mM. The presence of the canine domestic gene encoding the NADH core subunit 5: ubiquinone oxidoreductase (GenBank accession number: AAU12157.1) is determined after isothermal amplification at 65° C. for 30 min.

### 5.2 Results

TABLE 3
Determination of the effective concentrations of ZnSO₄ and KHCO₃ for
the extraction of nucleic acids from a dog blood sample

| ZnSO₄ conc. (mM)¹ | Amplification² | KHCO₃ conc. (mM)¹ | Amplification² |
|---|---|---|---|
| 25 mM | +(1/1) | 25 mM | +(1/1) |
| 33 mM | +(51/54) | 33 mM | +(1/1) |
| 42 mM | −(1/1) | 40 mM | +(51/54) |
| 43 mM | −(1/1) | | |
| 50 mM | −(1/1) | | |
| 59 mM | −(1/1) | | |
| 67 mM | −(1/1) | | |

¹Final concentration in the final reaction mixture comprising blood, the buffer comprising SDS, the buffer comprising ZnSO₄ and the buffer comprising KHCO₃ (total final volume before filtration 3 mL).
²Number of assays in which specific amplification of the domestic gene is observed over the total number of assays.

In conclusion, the final concentration of ZnSO₄ in the reaction mixture should be less than 42 mM, and that of KHCO₃ can be between about 25 mM and 40 mM, for extraction of nucleic acids from a blood sample of dog.

### Example 6: Extraction of Nucleic Acids from Various Samples

### 6.1 Materials and Methods

Blood and feces samples are taken according to the examples above.

Swabs and samples originating from nasopharyngeal and throat pouch lavages were collected according to standard medical or veterinary practice. Samples from environment were collected by rubbing a swab against a table (on a surface equivalent of a 25 cm² square) to collect all the particles available on this surface.

The presence of the feline domestic gene encoding the subunit 5 of NADH dehydrogenase (NDSS) (GenBank accession number: NC_001700.1); of the canine domestic gene encoding the NADH core subunit 5: ubiquinone oxidoreductase (GenBank accession number: AAU12157.1); of the equine domestic gene encoding (32 microglobulin (B2M gene) (GenBank accession number: AH011712.2) is determined after an isothermal amplification at 65° C. for 30 min.

The presence of the human domestic gene encoding the Homo sapiens ribonuclease P protein subunit p20 (RNAseP) (GenBank accession number: U94316.1) is determined after an isothermal amplification at 65° C. for 25 min.

Finally, the presence of a synthetic DNA sequence which does not exist in living organisms on earth was identified as a positive control. This synthetic DNA sequence was called “Alien”. The Alien sequence was synthetized and a small quantity of this DNA was added on the environment to be collected by the swab. The presence of a synthetic Alien DNA sequence in the environmental sample is determined after an isothermal amplification at 65° C. for 20 min

### 6.2 Results

The extraction conditions described hereunder in Table 4 and Table 5 all allowed significant amplification of the above-mentioned genes. This indicates that the extraction of nucleic acids from the complex samples was efficient.

TABLE 4
nucleic acid extraction from non-blood samples

| | | Potassium buffer |
|---|---|---|
| | Lysis buffer initial/final | initial/final |
| Sample’s origin | concentration | concentration |
| Oral/feces swab (cat) or | SDS: 5.0%/2.5% | KHCO₃: 0.4M/0.2M |
| Nasopharyngeal swab | | |
| (human) | | |
| Oral/feces swab (cat) or | SDS: 2.5%/1.22% | KHCO₃: 0.3M/0.146M |
| Nasopharyngeal swab | Tris-HCl 1M, pH | |
| (human) | 7.5/0.025M | |
| Throat Pouch lavage | SDS: 20.0%/2.4% | KHCO₃: 0.40M/0.21M |
| (horse) | | |
| Throat Pouch lavage | SDS: 20.0%/2.4% | KHCO₃: 0.30M/0.159M |
| (horse) | Tris-HCl 1M, pH | |
| | 7.5/0.023M | |
| Environmental swab | SDS: 5.0%/2.5% | KHCO₃: 0.4M/0.2M |

TABLE 5
nucleic acid extraction from blood samples

| | | Copper buffer | Potassium buffer |
|---|---|---|---|
| Sample’s | Lysis buffer initial/final | initial/final | initial/final |
| origin | concentration | concentration | concentration |
| Blood (cat/dog) | SDS: 5.0%/1.25% | CuSO₄ | KHCO₃: |
| | Tris-HCl 1M, pH | 1M/0.0375M | 0.3M/0.15M |
| | 7.5/0.025M | | |
| Blood (horse) | SDS: 5%/1.25% | CuSO₄ | KHCO₃: |
| | Tris-HCl 1M, pH | 1M/0.025M | 0.3M/0.15M |
| | 7.5/0.025M | | |

As a conclusion, the initial concentrations of SDS, and KHCO₃ within their respective buffers may vary without affecting the nucleic acids' extraction yield. In addition, the presence of Tris-HCl was shown to improve the yield of extracted nucleic acids.

Finally, KHCO₃ could be replaced with K₂HPO₄ or KH₂PO₄ without affecting the extraction (see Table 6 below).

TABLE 6
nucleic acid extraction from non-blood samples with various
potassium salts

| | | Potassium buffer |
|---|---|---|
| | Lysis buffer initial/final | initial/final |
| Sample’s origin | concentration | concentration |
| Oral swab (cat) or | SDS: 2.5%/1.22% | KHCO₃: |
| Nasopharyngeal swab | Tris-HCl 1M, pH | 0.3M/0.146M |
| (human) | 7.5/0.025M | |
| Oral swab (cat) or | SDS: 2.5%/1.22% | KH₂PO₄: |
| Nasopharyngeal swab | Tris-HCl 1M, pH | 0.3M/0.146M |
| (human) | 7.5/0.025M | |
| Oral swab (cat) or | SDS: 2.5%/1.22% | K₂HPO₄: |
| Nasopharyngeal swab | Tris-HCl 1M, pH | 0.15M/0.073M |
| (human) | 7.5/0.025M | |

1.-15. (canceled)

16. A kit for extracting nucleic acids contained in a complex sample, said kit comprising:
a lysis buffer comprising a concentration of SDS ranging from about 1% to about 25%, preferably from about 2.5% to about 20%;
a buffer comprising a concentration of a potassium salt ranging from about 0.1 M to about 5.0 M;
a buffer comprising a concentration of a zinc and/or copper salt ranging from about 0.5 M to about 5.0 M;
a filter having a pore diameter ranging from about 1 μm to about 10 μm, preferably from about 2 μm to about 7 μm;
optionally, an item chosen from the group comprising or consisting of one more syringe(s), one or more reaction tube(s), an instruction guide, and any combination thereof.

17. The kit according to claim 16, wherein the zinc salt is selected from a group comprising or consisting of zinc sulfate (ZnSO₄), and zinc chloride (ZnCl₂).

18. The kit according to claim 16, wherein the copper salt is selected from a group comprising or consisting of copper sulfate (CuSO₄), and copper chloride (CuCl₂).

19. The kit according to claim 16, wherein the potassium salt is selected from the group comprising or consisting of potassium hydrogen carbonate (KHCO₃), potassium acetate (CH₃CO₂K), dipotassium hydrogen phosphate (K₂HPO₄), monobasic potassium phosphate (KH₂PO₄), and potassium chloride (KCl).

20. The kit according to claim 16, wherein the lysis buffer and/or the buffer comprising a zinc and/or copper salt comprise(s) one or more additional compound(s) selected in the group comprising or consisting of Tris-HCl, HEPES, and MOPS.

21. A method for extracting nucleic acids contained in a complex sample, comprising extracting the nucleic acid using the kit according to claim 16.

22. A method for extracting nucleic acids contained in a complex sample, said method comprising the following steps:
a) contacting the complex sample with a lysis buffer comprising a concentration of SDS ranging from about 1% to about 25%, so that the final concentration of SDS in the reaction mixture is ranging from about 0.01% to about 10.0%;
b) optionally contacting the mixture from step a) with a buffer comprising a concentration of a zinc and/or copper salt ranging from about 0.5 M to about 5.0 M, so that the final salt concentration zinc and/or copper in the reaction mixture is ranging from about 10 mM to about 70 mM;
c) contacting the mixture of step a) or b) with a buffer comprising a concentration of a potassium salt ranging from about 0.1 M to about 5.0 M, so that the final concentration potassium salt in the reaction mixture is ranging from about 10 mM to about 500 mM;
d) filtering the reaction mixture from step c); and
e) collecting soluble nucleic acids.

23. The method according to claim 22, wherein the complex sample is selected from the group comprising a biological sample, environmental sample, food sample, preferably a biological sample.

24. The method according to claim 23, wherein the biological sample is blood.

25. The method according to claim 24, wherein step b) is performed when the complex sample is a blood sample.

26. The method according to claim 24, wherein the final concentration of SDS in the reaction mixture is ranging from about 0.01% to about 2%, preferably from about 0.01% to about 1.5%, preferably from about 0.01% to about 1%, preferably from about 0.25% to about 0.75%, when the complex sample is a blood sample.

27. The method according to claim 24, wherein the final concentration of potassium salt in the reaction mixture is ranging from about 10 mM to about 500 mM, preferably from about 25 mM to about 150 mM, when the complex sample is a blood sample.

28. The method according to claim 23, wherein the biological sample is selected from the group consisting of feces sample, saliva sample, a product of respiratory lavage sample, and a nasopharyngeal secretion sample.

29. The method according to claim 28, wherein the final concentration of SDS in the reaction mixture is ranging from about 0.06% to about 10%, preferably from about 0.1% to about 6.7%, when the complex sample is selected from group consisting of a feces sample, a saliva sample, a respiratory lavage sample, and a nasopharyngeal secretion sample.

30. The method according to claim 28, wherein the final concentration of potassium salt in the reaction mixture is ranging from about 60 mM to about 300 mM, preferably from about 100 mM to about 270 mM, when the complex sample is selected from the group consisting of a feces sample, a saliva sample, respiratory lavage sample, and a nasopharyngeal secretion sample.
